(19) United States
(12) Patent Application Publication
(10) Pub. No.: US 2004/0234581 A1
(43) Pub. Date: Nov. 25, 2004

(54) Disposable facial mask

(76) Inventor: Douglas F. Souther, Phonenix, AZ (US)

Correspondence Address: Steven Thrasher, 391 Sandhill, Richardson, TX, 75080, US

(21) Appl. No.: 10/443,975

(22) Filed: May 23, 2003

(51) Int. Cl. A61K 9/70

(52) U.S. Cl. 424/443

(57) ABSTRACT

The invention is a disposable, single-use, medicated facial mask (the mask). The mask includes a medicated portion adapted to pass a sufficient amount of air to allow a person to breath normally, and also adapted to filter particles from the air. The mask also has a skin-contact portion disposed adjacent to the medicated portion and adapted to fit securely about a human face.

### TECHNICAL FIELD OF THE INVENTION

The invention relates generally to medical supplies, and, more particularly, the invention relates to devices that slow the spread of communicable diseases.

### STATEMENT OF A PROBLEM ADDRESSED BY THIS INVENTION

Interpretation Considerations

This section describes the technical field in more detail, and discusses problems encountered in the technical field. This section does not describe prior art as defined for purposes of anticipation or obviousness under 35 U.S.C. section 102 or 35 U.S.C. section 103. Thus, nothing stated in the Statement of a Problem Addressed by This Invention is to be construed as prior art

Discussion

As society becomes more interactive, there has been a rise in the spread of communicable diseases. Evidence exists that Sever Acute Repertory Syndrome (SARS) originated from an exotic animal market in a remote region of China, and then spread to Toronto, Canada, in just a matter of a few days. SARS infected over 150 persons in Toronto, killing 15 of them, prior to being contained.

Every day in work places, schools, hospitals, and doctor offices persons with air-born communicable diseases expose dozens, if not hundreds of other people to their disease. This can cause an outbreak of the flue, influenza, or other diseases, which may quickly spread to infect thousands in a local population.

Illnesses cause increased worker absenteeism, missed class days, liability issues, and even hospitalization or death. In addition to illness, the spread of communicable disease brings social consequences. For example, the threat of catching SARS sent Hong Kong into a recession in early 2003. Similarly, often persons change their travel plans or buying patterns to avoid catching a disease. To avoid catching air-born pollen, or viruses and bacteria that cause disease, many persons wear a facial mask.

Though generally effective against allergens, facial mask have proven less effective against preventing the transmission of viral and micro-bacterial pathogens. Accordingly, there is needed a device for reducing the likelihood of the transmission of an air-born communicable disease.

### Selected Overview of Selected Embodiments

The invention provides technical advantages as a disposable, single-use, medicated facial mask (the mask). The mask includes a medicated portion adapted to pass a sufficient amount of air to allow a person to breath normally, and also adapted to filter particles from the air. In a preferred embodiment, the medicated portion comprises an anti-viral or anti-microbial substance, such as iodine. The mask also has a skin-contact portion disposed adjacent to the medicated portion and adapted to fit securely about a human face.

Of course, other features and embodiments of the invention will be apparent to those of ordinary skill in the art. After reading the specification, and the detailed description of the exemplary embodiment, these persons will recognize that similar results can be achieved in not dissimilar ways. Accordingly, the detailed description is provided as an example of the best mode of the invention, and it should be understood that the invention is not limited by the detailed description. Accordingly, the invention should be read as being limited only by the claims.

### BRIEF DESCRIPTION OF THE DRAWINGS

Various aspects of the invention, as well as at least one embodiment, are better understood by reference to the following EXEMPLARY EMBODIMENT OF A BEST MODE. To better understand the invention, the EXEMPLARY EMBODIMENT OF A BEST MODE should be read in conjunction with the drawings in which:

FIG. 1 illustrates a medicated facial mask system; and

FIG. 2 shows one embodiment of a medicated facial mask.

### AN EXEMPLARY EMBODIMENT OF A BEST MODE

The invention is a disposable, single-use, medicated facial mask (the mask). The mask includes a medicated portion adapted to pass a sufficient amount of air to allow a person to breath normally, and also adapted to filter particles from the air. The mask also has a skin-contact portion disposed adjacent to the medicated portion and adapted to fit securely about a human face.

Interpretation Considerations

When reading this section (An Exemplary Embodiment of a Best Mode, which describes an exemplary embodiment of the best mode of the invention, hereinafter “exemplary embodiment”), one should keep in mind several points. First, the following exemplary embodiment is what the inventor believes to be the best mode for practicing the invention at the time this patent was filed. Thus, since one of ordinary skill in the art may recognize from the following exemplary embodiment that substantially equivalent structures or substantially equivalent acts may be used to achieve the same results in exactly the same way, or to achieve the same results in a not dissimilar way, the following exemplary embodiment should not be interpreted as limiting the invention to one embodiment.

Likewise, individual aspects (sometimes called species) of the invention are provided as examples, and, accordingly, one of ordinary skill in the art may recognize from a following exemplary structure (or a following exemplary act) that a substantially equivalent structure or substantially equivalent act may be used to either achieve the same results in substantially the same way, or to achieve the same results in a not dissimilar way.

Accordingly, the discussion of a species (or a specific item) invokes the genus (the class of items) to which that species belongs as well as related species in that genus. Likewise, the recitation of a genus invokes the species known in the art. Furthermore, it is recognized that as technology develops, a number of additional alternatives to achieve an aspect of the invention may arise. Such advances are hereby incorporated within their respective genus, and should be recognized as being functionally equivalent or structurally equivalent to the aspect shown or described.

Second, the only essential aspects of the invention are identified by the claims. Thus, aspects of the invention, including elements, acts, functions, and relationships (shown or described) should not be interpreted as being essential unless they are explicitly described and identified as being essential. Third, a function or an act should be interpreted as incorporating all modes of doing that function or act, unless otherwise explicitly stated (for example, one recognizes that “tacking” may be done by nailing, stapling, gluing, hot gunning, riveting, etc., and so a use of the word tacking invokes stapling, gluing, etc., and all other modes of that word and similar words, such as “attaching”). Fourth, unless explicitly stated otherwise, conjunctive words (such as “or”, “and”, “including”, or “comprising” for example) should be interpreted in the inclusive, not the exclusive, sense. Fifth, the words “means” and “step” are provided to facilitate the reader's understanding of the invention and do not mean “means” or “step” as defined in §112, paragraph 6 of 35 U.S.C., unless used as “means for—functioning—” or “step for—functioning—” in the Claims section.

Discussion of the Figures

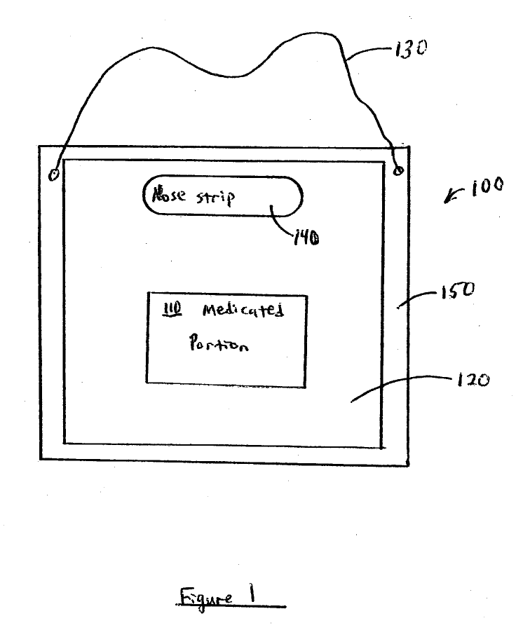

Features and advantages of the invention can be better understood by reviewing FIG. 1, which illustrates a medicated facial mask system (the facial mask system 100). The facial mask system generally includes a medicated portion 110 that carries an anti-viral and/or an anti-microbial compound, and a skin contact portion 120. The facial mask is disposable, and is preferably replaced after a definable time frame based on preserving the efficacy of the anti-viral and/or anti-microbial compound.

The medicated portion 110 is preferably adapted to pass a sufficient amount of air to allow a person to breath normally, and also adapted to filter particles from the air. Filtered particles include pollens, and preferably includes viruses and microbial germs. However, it is appreciated by those of ordinary skill in the art that some viruses and organisms are too small to be reliably removed from air by a filter alone. Accordingly, the medicated portion provides at least one anti-viral and/or an anti-microbial compound, and preferably includes iodine. Thus, the communication of a disease is reduced since healthy individuals using the facial mask system 100 breath “treated” air, and because sick (infected) individuals treat the air they exhale so that there are fewer germs for healthy individuals to breath.

The skin-contact portion 120 is disposed adjacent to the medicated portion, and is adapted (such as through shape) to fit securely about a human face. The skin contact 120 preferably does not itself facilitate airflow (and, in fact, is preferably not air-permeable), and preferably promotes the flow of air through the medicated portion 110. The skin contact portion 120 is also preferably not medicated, but instead is not treated with medication, thus preventing undesired medications from being absorbed through the skin. In addition, the skin contact portion 120 may comprise vitamins or other beneficial compounds that are skin-absorbable.

A strap 130 is coupled to the skin-contact portion 120. The strap 130 is preferably adapted to secure the facial mask system 100 about a head (not shown). In addition or alternatively, a nose-strip 140 is coupled to the skin-contact portion 120, and is adapted to secure the facial mask system 100 to a face. In an alternative embodiment, an adhesive surface 150 is disposed about the outside perimeter of the skin-contact portion 120. The adhesive surface 120 is adapted to secure the facial mask system 100 to a face by reducing the amount of “untreated air” that enters the facial mask system 100 or exits the facial mask system 100 via the medicated portion 110. Generally, facial mask straps and nose strips are well known to those of ordinary skill in the art.

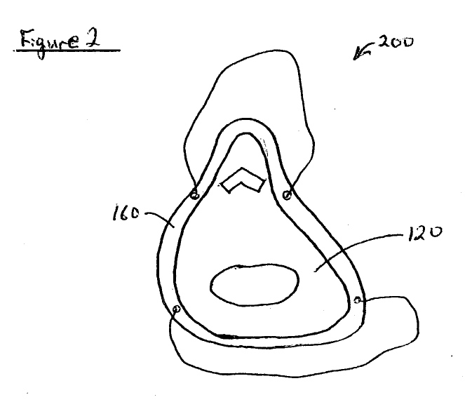

FIG. 2 shows one embodiment of a medicated facial mask 200. Like numerals represent like systems and inventive elements unless otherwise stated. The medicated facial mask 200 includes the medicated portion 110, the skin contact portion 120, strap 130 and nose strip 140 of the facial mask system 100 from the facial mask system 100 of FIG. 1. In addition, the medicated facial mask 200 includes a comfort fitting 160 coupled to the skin-contact portion 120, and being disposed about the perimeter of the skin-contact portion 120. From FIG. 2 one can see that the comfort fitting 160 is adapted to comfortably take the shape of a face. Of course, different cultures and individuals have differently shaped faces and the medicated facial mask 200 can take any size and shape desired to facilitate attachment to a face. In one embodiment, advantages are provided by a comfort fitting 160 that is adapted to be moisture absorbent, such as through the use of cotton or other fabrics, or through the use of absorbent materials, such as cornstarch. In an alternative embodiment, the invention provides advantages via a comfort fitting 160 that is adapted to feel soft on a face through the use of fabric, cotton, or other soft material. Yet additional advantages can be provided via a comfort fitting 160 adapted to be non-chafing to minimize rubbing. In addition, it will be appreciated that a user can sanitize fingers by rubbing them on a treated area of the mask.

Thus, though the invention has been described with respect to a specific preferred embodiment, many variations and modifications will become apparent to those skilled in the art upon reading the present application. It is therefore the intention that the appended claims be interpreted as broadly as possible in view of the prior art to include all such variations and modifications.

What is claimed is:

1. A disposable, single-use, medicated facial mask, comprising:
a medicated portion, the medicated portion adapted to pass a sufficient amount of air to allow a person to breath normally, and also adapted to filter particles from the air; and
a skin-contact portion disposed adjacent to the medicated portion, the skin-contact portion adapted to fit securely about a human face.

2. The facial mask of claim 1 wherein the medicated portion comprises iodine.

3. The facial mask of claim 1 wherein the medicated portion comprises an antiviral compound.

4. The facial mask of claim 1 wherein the medicated portion comprises an anti-bacterial compound.

5. The facial mask of claim 1 further comprising a strap coupled to the skin-contact portion, the strap adapted to securely strap the facial mask about a head.

6. The facial mask of claim 1 further comprising a nose-strip coupled to the skin-contact portion, the nose-strip adapted to securely couple the facial mask to a face.

7. The facial mask of claim 1 further comprising an adhesive surface disposed about the perimeter of the skin-contact portion, the adhesive surface being adapted to securely fit the facial mask to a face.

8. The facial mask of claim 1 further comprising a comfort fitting coupled to the skin-contact portion, and being disposed about the perimeter of the skin-contact portion, the comfort fitting being adapted to comfortably take the shape of a face.

9. The facial mask of claim 8 wherein the comfort fitting is adapted to be moisture absorbent.

10. The facial mask of claim 8 wherein the comfort fitting is adapted to feel soft on a face.

11. The facial mask of claim 8 wherein the comfort fitting is adapted to be non-chafing.

12. A disposable, single-use, medicated facial mask, comprising:
a medicated portion, the medicated portion adapted to pass a sufficient amount of air to allow a person to breath normally, and also adapted to filter particles from the air, the medicated portion comprising iodine; and
a skin-contact portion disposed adjacent to the medicated portion, the skin-contact portion adapted to fit securely about a human face.

13. A disposable, single-use, medicated facial mask, comprising:
a medicated portion, the medicated portion adapted to pass a sufficient amount of air to allow a person to breath normally, and also adapted to filter particles from the air, the medicated portion comprising iodine;
a skin-contact portion disposed adjacent to the medicated portion, the skin-contact portion adapted to fit securely about a human face; and
a strap coupled to the skin-contact portion, the strap adapted to securely strap the facial mask about a head.
